(19) United States
(12) Patent Application Publication
(10) Pub. No.: US 2026/0284372 A1
(43) Pub. Date: Sep. 24, 2026

(54) MICRONEEDLE PACKAGE AND APPLICATOR PACKAGE COMPRISING SAME

(71) Applicant: CGBIO CO., LTD., Seoul (KR)

(72) Inventors: Chan Woo HONG, Incheon (KR); Hyun A PARK, Seongnam-si (KR)

(21) Appl. No.: 19/164,177

(22) PCT Filed: Sep. 19, 2023
(86) PCT No.: PCT/KR2023/014106

§ 371 Date: Sep. 10, 2025

(30) Foreign Application Priority Data
Mar. 13, 2023 (KR) 10-2023-0032286

(51) Int. Cl. A61M 37/00

(52) U.S. Cl. CPC A61M 37/0015; A61M 2207/00; A61M 2209/06

(57) ABSTRACT

The present disclosure relates to a microneedle package and an applicator package including the microneedle package. One embodiment may comprise: a needle protection part in which microneedles are disposed; and a case in which the needle protection part is disposed and which guides an applicator so as to be seated on the microneedles.

### CROSS-REFERENCE TO RELATED APPLICATIONS

This application is the National Stage filing under 35 U.S.C. 371 of International Application No. PCT/KR2023/014106, filed on Sep. 19, 2023, which claims the benefit of Korean Patent Application No. 10-2023-0032286, filed on Mar. 13, 2023, the contents of which are all hereby incorporated by reference herein in their entirety.

### TECHNICAL FIELD

The present disclosure relates to a microneedle package and an applicator package including the microneedle package.

### BACKGROUND ART

Microneedles or micro-pins have small needle-shaped structures disposed therein, so they are used for the delivery of therapeutic agents and other substances through the skin and other surfaces. Microneedles are used in such a shape that they are pressed against a surface, particularly the skin, so that the therapeutic agents and other substances enter the underlying tissues sequentially or simultaneously through the surface such as the skin or the like. At this time, microneedles can penetrate the stratum corneum and deliver active ingredients into the body in a variety of ways. For example, hollow microneedles containing a reservoir to temporarily hold the active ingredients can penetrate the stratum corneum and deliver the active ingredients into the body, and since the microneedles are coated with the active ingredients, the microneedles can penetrate the stratum corneum and deliver the active ingredients into the body. In addition, soluble microneedles can penetrate the stratum corneum to deliver the active ingredients while being dissolved within the body.

Drug delivery using soluble microneedles is greatly affected not only by the direct mechanochemical properties of the microneedle including shape, physical properties, and solubility, but also by the application method such as the administration (insertion) state and administration time. Therefore, if the appropriate application method is not adopted for administration, not only may individual needles not be properly inserted into the dermal layer, but the fixed amount of drug may not be delivered. Furthermore, since there is a case that separate adhesive means such as pressure-sensitive adhesive sheets or adhesive paper are used in order to attach soluble microneedles to the skin and secure them in place for a long time, there have been difficulties in applying and attaching them to joints with a lot of movement, curved skin, or hairy skin.

To improve these problems, methods for administering microneedles using microneedle applicators have been disclosed, but there have been problems in that conventional microneedle applicators have complicated microneedle mounting methods and are vulnerable to damage or contamination of the microneedles when the microneedles are mounted on the applicators.

Furthermore, since a phenomenon that microneedles applied to applicators bend has been conventionally occurred, there has been a problem in that the microneedles are broken due to higher needle heights compared to cosmetic microneedles when used in pharmaceuticals or veterinary medicines.

Therefore, there is a need for a microneedle package that can prevent damage and contamination of microneedles and improve usability, and an applicator package that includes the microneedle package.

### DISCLOSURE

### Technical Problem

In order to solve the above-described problems, the present disclosure aims to provide a microneedle package that can prevent damage and contamination of a microneedle and improve usability, and an applicator package including the microneedle package.

In addition, the present disclosure aims to provide a microneedle package that can form the microneedle vertically from an adhesive means without the microneedle being bent when the microneedle is separated from the microneedle package by an applicator, and an applicator package including the microneedle package.

In addition, the present disclosure aims to provide a microneedle package that allows the microneedle to be formed in a shape corresponding to a microneedle forming part provided in a mold without the microneedle being damaged when the microneedle is separated from the microneedle package by an applicator, and an applicator package including the microneedle package.

In addition, the present disclosure aims to provide a microneedle package that has a high degree of reliability in the quality of the formed microneedle, and an applicator package including the microneedle package.

### Technical Solution

To address the above-described problems, the present disclosure provides a microneedle package including: a needle protection part in which a microneedle is disposed; and a case in which the needle protection part is disposed and which guides an applicator to be seated on the microneedle.

For example, the needle protection part may include a case fastening part in the form of extending rearward.

For example, the needle protection part further includes a first member provided with the case fastening part and a second member for being coupled to the rear of the first member, and the case fastening part allows the position of the first member to be fixed at a predetermined position within the case, and the first member may move forward by an operation in which the case fastening part is pressed by the applicator.

At this time, the case fastening part is provided with three pressed regions that are pressed by the applicator, and the first member is pressed by the applicator so that the first member has a surface perpendicular to the applicator, thereby forming the microneedle vertically.

At this time, the needle protection part further includes a first member and a second member to be coupled at the rear of the first member, and the first member, whose position is in a fixed state within the case, can be vertically moved forward by the operation of the applicator simultaneously pressing a plurality of the case fastening parts forward.

At this time, the microneedle may be vertically separated from the first member by an operation of the first member moving vertically forward.

For example, the needle protection part further includes a first member and a second member for being coupled to the rear of the first member, and the second member is provided in a state coupled to the first member, but when applied to the applicator, the microneedle is separated from the first member in a state in which the microneedle is attached to the second member, thereby preventing contamination and damage to the microneedle before use and improving convenience of use.

At this time, the first member and the second member are installed within a case extending in the longitudinal direction to have a predetermined length, but the second member is seated on a stepped projection provided at the bottom of the case, and the first member may be detachably fitting-coupled to be fixed inside the case, thereby overlappingly installing the second member and the first member within the case.

At this time, when the applicator is inserted into the case, the second member is fastened to the front portion of the applicator in a state in which the second member is seated on the stepped projection, and the first member may move to the bottom surface of the case by pressing of the applicator.

For example, the needle protection part may be provided with a mold mounting part for seating the mold, and the mold may be provided with a microneedle forming part recessed to a predetermined depth on the rear surface.

At this time, the microneedle forming part may include a base forming part and a plurality of needle forming parts extending forward from the base forming part.

Meanwhile, the adhesive means having adhesive force is disposed forwardly on the second member, the adhesive means includes: a needle attachment part which is formed in the center and is for adhering the microneedle; a second member attachment part which is defined as a region where the microneedle is seated on the second member; and an adhesive means cutting line which is defined as an empty region formed between the needle attachment part and the second member attachment part, wherein the needle attachment part may be separated from the second member attachment part when the microneedle is fired toward the target region.

At this time, the second member includes: a guide member extending to the rear of the second member to define a space that guides the approach of the applicator; and a step protruding to the inside of the guide member at a predetermined height and extending to the front of the second member to define a region into which the first member is inserted, and the second member attachment part may be disposed on a step surface of the step.

At this time, the guide member may be divided into a plurality of pieces to impart elasticity when the applicator is inserted, thereby imparting a fixing force to the applicator.

For example, the first member may be provided with a plurality of case fastening parts that are in the form of extending rearward, and guide grooves for inserting the case fastening parts into the case while the case fastening parts are being guided through the guide grooves may be provided on the inner side of the case, thereby easily installing the first member into the case, and restricting rotation of the first member after installation.

At this time, the second member includes a plurality of tongue parts which protrude outwardly so that the applicator is fastened to the tongue parts; and a case fastening part guide part defined as a space which is provided between the tongue parts and into which the case fastening parts are inserted, wherein rotation of the second member from the first member may be restricted by the case fastening part guide part.

In addition, in the aforementioned microneedle package of the present disclosure, the needle protection part in which the microneedle is disposed may be provided in a state in which the needle protection part is stored in a sealed space within the case, thereby preventing the inflow of foreign materials and preventing contamination of the microneedle prior to use.

Furthermore, the present disclosure may provide an applicator package including: the aforementioned microneedle package; and an applicator coupled to the rear of the microneedle package.

At this time, during an operation of coupling the applicator to and then detaching it from the microneedle package, the second member is separated from the microneedle package and fastened to the applicator, but the microneedle may be provided in front of the needle protection part.

Meanwhile, the applicator may include a pressing part that is at least partially inserted into the applicator and moves along the longitudinal direction of the applicator, and the rear surface of the microneedle may be disposed in front of the pressing part.

At this time, the applicator further includes a slide that is disposed at the front portion of the applicator and moves in the longitudinal direction, and the slide may be operated in conjunction with the pressing part.

At this time, the applicator may further include a linear guide provided at the side portion to allow the slide to move in the longitudinal direction.

At this time, the case may further include protrusion pieces in such a form that they protrude to the inner side of the case, but extend in the longitudinal direction.

Meanwhile, when the applicator is inserted into the case, the slide moves rearward of the applicator due to pressure applied by the protrusion pieces; the pressing part moves rearward of the applicator in conjunction with the movement of the slide; the slide and the pressing part that move rearward are fixed at a predetermined position; and after the slide and the pressing part are fixed at a predetermined position, the slide and the pressing part move forward so that the microneedle may be separated from the needle protection part.

### Advantageous Effects

The present disclosure can prevent contamination and damage to the microneedle by providing a microneedle package in which an adhesive means and a first member within a case protect a microneedle and allows the microneedle to be blocked from the external environment.

In addition, the present disclosure provides an applicator package which includes the microneedle package and an applicator capable of extracting a microneedle by inserting and separating motions for the microneedle package, thereby allowing continuous reuse of the applicator to enable convenience of use to be improved.

In addition, in the present disclosure, the case is formed to a predetermined length, a mold, a microneedle, and an adhesive means are disposed perpendicular to the longitudinal direction of the case, so the microneedle can be formed in a direction perpendicular to the adhesive means without being bent when the microneedle is separated from the microneedle package by the applicator.

In addition, in the present disclosure, when the applicator is inserted into the case of the microneedle package, the applicator can be allowed to be inserted perpendicularly to the microneedle by guide means provided in the case and the microneedle package, thereby allowing the microneedle to be formed in a direction perpendicular to the front surface of the applicator without being bent.

In addition, in the present disclosure, two or more slides are provided on the side portion of an applicator, and two or more guide means corresponding thereto, for example, protrusion pieces, are provided inside the case so that the applicator can be inserted vertically into the microneedle, thereby enabling the microneedle to be formed in a direction perpendicular to the front surface of the applicator without being bent.

In addition, in the present disclosure, a structure in which the applicator moves longitudinally within the microneedle package case is applied so that when the microneedle is separated from the microneedle package by the applicator, the microneedle can be formed in a shape corresponding to the microneedle forming part provided in the mold without being damaged.

In addition, in the present disclosure, when the second member is coupled to the applicator, a motion in which the needles are separated from the mold and a motion in which the position is fixed in a state in which the pressing part is pulled backward are accompanied, thereby enabling the microneedle to be formed in a direction perpendicular to the front surface of the applicator without being bent.

### DESCRIPTION OF DRAWINGS

FIG. 1 shows a microneedle package according to an embodiment of the present disclosure.

FIGS. 2, 3, and 4 show embodiments in which a microneedle is formed and separated in a mold according to an embodiment of the present disclosure.

FIGS. 5 and 6 show a first member and a second member according to an embodiment of the present disclosure.

FIGS. 7 and 8 show embodiments in which a microneedle cartridge and a second member are separated therefrom according to an embodiment of the present disclosure.

FIGS. 9, 10, and 11 show embodiments in which a microneedle cartridge is mounted and separated from a case in a microneedle package according to an embodiment of the present disclosure.

FIGS. 12, 13, and 14 show an applicator package including an applicator and a microneedle package according to an embodiment of the present disclosure.

FIGS. 15, 16, 17, 18, 19, and 20 show embodiments of the applicator package according to an embodiment of the present disclosure, according to the order in which the microneedle is brought into contact with the skin, fired, and detached, wherein FIGS. 15, 16, and 17 show shapes of the applicator package, and FIGS. 18, 19, and 20 show lower portions of the applicator package, respectively.

FIGS. 21 and 22 show embodiments in which the needle attachment part is separated from the second member according to an embodiment of the present disclosure.

FIGS. 23 and 24 show embodiments in which the second member is separated from the applicator according to an embodiment of the present disclosure.

FIGS. 25 and 26 show shapes of the microneedle, wherein FIG. 25 shows a microneedle manufactured according to one embodiment of the present disclosure, and FIG. 26 shows a conventionally manufactured microneedle.

### MODE FOR DISCLOSURE

Hereinafter, embodiments of the present disclosure will be described in detail. However, the present disclosure is not limited to the embodiments disclosed below and may be implemented in various other forms, and these embodiments are provided solely to ensure the complete disclosure of the present disclosure and to more fully convey the content of the present disclosure to those skilled in the art.

When one element is referred to as being positioned “above” or “below” another element in this specification, this includes both a meaning that the one element is directly positioned “above” or “below” the other element, or a meaning that additional elements can be interposed between those elements.

In this specification, the terms “upper” and “lower” are relative concepts established from the observer's perspective, and if the observer's perspective changes, “upper” may mean “lower,” and “lower” may mean “upper.” In this specification, the terms “top” and “bottom” are relative concepts established from the observer's perspective, and if the observer's perspective changes, “top” may mean “bottom,” and “bottom” may mean “top.” In this specification, “front direction” or “front” refers to the direction in which the applicator is attached to the skin, “rear direction” or “rear” refers to the opposite direction, and “front” refers to the surface formed in the front, and “rear” refers to the surface formed in the opposite direction.

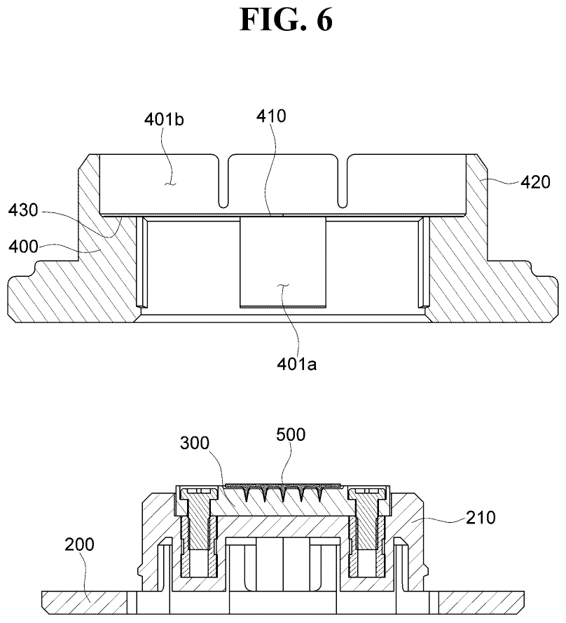

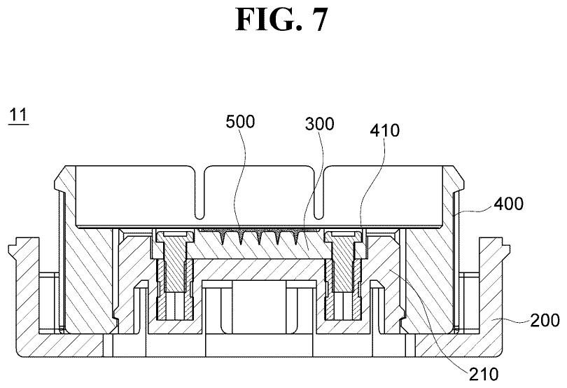

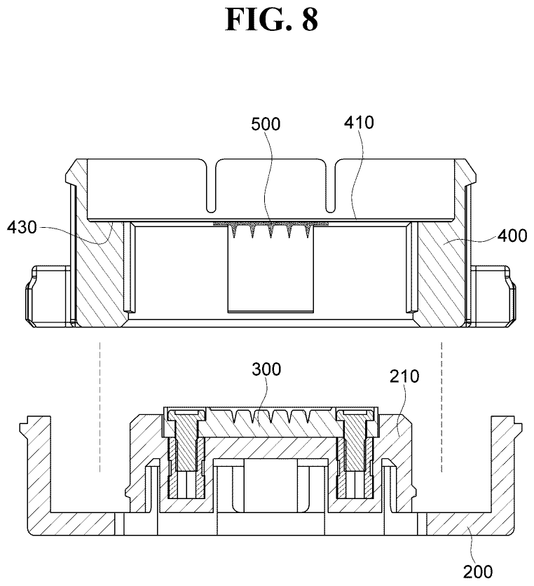

Unless otherwise specifically limited in this specification, “length direction” refers to the direction in which the applicator enters the case, and “thickness direction” refers to the direction perpendicular to the “length direction.” The applicator package referred to in this specification includes the operational states of coupling and disengaging the microneedle package and applicator described in the first through sixth operational states illustrated with reference to FIGS. 6 and 7.

This will be described in detail below with reference to the drawings.

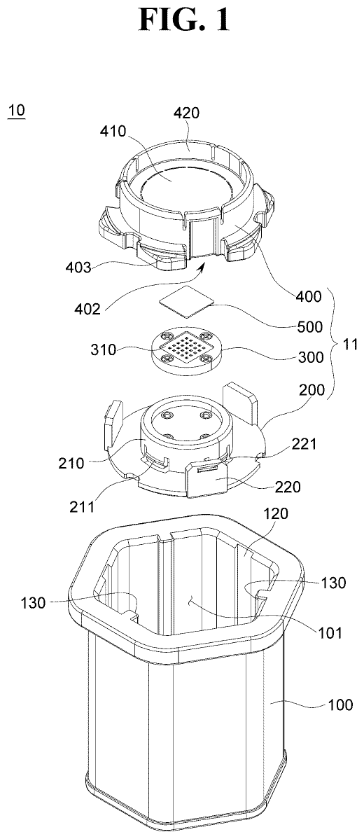

FIG. 1 shows a microneedle package according to an embodiment of the present disclosure.

Referring to FIG. 1, the microneedle package 10 may include a case 100 and a microneedle cartridge 11.

The case 100 may have one surface opened and the other surface corresponding thereto closed, and may be in the form of a cup with the one surface and the other surface being connected longitudinally, and a case opening 101 may be formed in the one surface. The microneedle cartridge 11 may be inserted and mounted into the case 100 through the case opening 101.

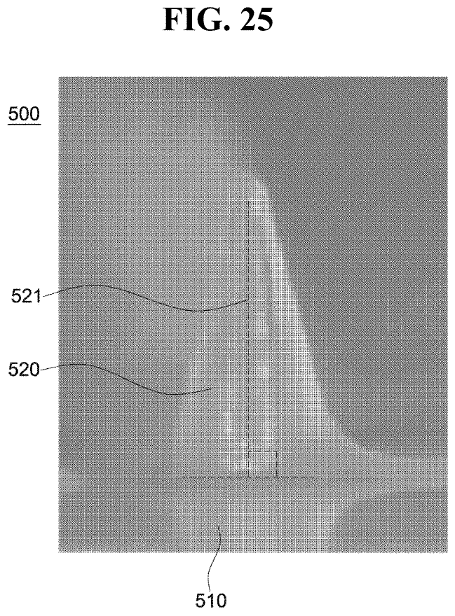

For example, the case 100 is formed to a predetermined length so that the applicator, which will be described later, may be inserted and separated by moving to a predetermined length. At this time, the movement of the applicator is guided to move in one direction so that when the microneedle is separated from the microneedle package by the applicator, the microneedle may be formed in a vertical direction from the adhesive means without being bent. At this time, the meaning of being formed in the vertical direction includes at least one of the following: the center line 521 of the needle 520 is formed in a shape of extending vertically from the base 510, as illustrated in FIG. 25, and the cross-sectional area is formed to gradually narrow at a predetermined ratio as it extends from the base 510, and the same applies in the following description. For example, a guide means for guiding the introduction of the applicator may be provided inside the case 100. For example, protrusion pieces 130 which have a predetermined length and are in an extended form may be provided inside the case 100. The protrusion pieces 130 perform functions of guiding the vertical directional insertion of the applicator 20, which will be described later, and applying pressure to the slide 22 of the applicator 20 to stably push the slide 22 upward at the same time. Simultaneously, the protrusion pieces perform a function of preventing the applicator 20 from rotating when inserted into the case 100.

The microneedle cartridge 11 may include a needle protection part 12 and a microneedle 500.

The shape and function of the needle protection part 12 are not particularly limited as long as the microneedle 500 is disposed therein.

For example, the needle protection part 12 may be composed of a single member or multiple members.

For example, the needle protection part 12 may include a first member 200 and a second member 400.

The microneedle 500 may be formed on the first member 200, and, for example, a mold 300 may be disposed on the first member 200.

At this time, a mold mounting part 210 may be provided at the center of the first member 200 for mounting the mold 300.

The mold mounting part 210 may include a shape corresponding to the shape of the mold 300 so that the mold 300 may be mounted and secured. For example, the mold mounting part 210 may include a recessed region for seating the mold 300 and securing its movement.

In the applicator package described later, when the mold 300 is separated after being coupled to the applicator, the mold 300 is remained on the first member 200, and the microneedle 500 may be stably vertically separated from the mold 300 together with the adhesive means 410.

For example, the mold 300 is a single member manufactured integrally with the first member 200, and the term may be distinguished from the first member 200 only to refer to the region where the microneedle 500 is formed.

For example, the mold 300 may be a member that can be separated from the first member 200 and fastened thereto. Various methods such as fastening means such as screws, members with magnetic properties, adhesive means, and fitting coupling by elasticity may be applied as the fastening method.

The mold mounting part 210 may have a predetermined area and a shape of extending rearward from the first member 200. At this time, an elastic piece 211 protruding in the thickness direction may be formed on the side of the mold mounting part 210. The elastic piece 211 is applied to fasten the first member 200 to the second member 400 and separate the first member 200 from the second member 400.

For example, when the microneedle cartridge 11 is configured in a state in which the first member 200 and the second member 400 are coupled, at least a portion of the mold mounting part 210 may be provided in a state in which it is inserted into the second member 400. At this time, the elastic piece 211 may be fitting-coupled to the inner side of the second member 400 so that the second member 400 does not separate from the first member 200 within the case 100 constituting the microneedle package 10. Meanwhile, the fitting coupling of the elastic piece 211 to the inner side of the second member 400 may be released by the withdrawal force of separating after coupling the applicator described later to the microneedle package 10, thereby separating the second member 400 from the first member 200.

Accordingly, the microneedle 500 may be protected and blocked from the external environment, thereby preventing contamination and damage to the microneedle and simultaneously improving convenience of use, and organic coupling with other components of the present disclosure may allow the applicator to be inserted in a vertical direction, and the microneedle may be formed in a vertical direction from the adhesive means without being bent.

The first member 200 may have a case fastening part 220 formed at a thickness direction end portion.

For example, the case fastening part 220 may have a shape of extending rearward.

For example, the case fastening part 220 may secure the position of the first member 200 at a predetermined position within the case 100, but the first member 200 may move forward by a motion of pressing the case fastening part 220 by the applicator.

For example, the case fastening part 220 may be provided with three pressed regions that are pressed by the applicator, and the first member 200 may be pressed by the applicator so that it has a surface perpendicular to the applicator, thereby allowing the microneedle 500 to be formed vertically.

At this time, if only one or a portion of the three pressed regions are pressed by the applicator, the position of the first member 200 may be maintained in a fixed state within the case 100, or the microneedle 500 may not be separated from the first member 200.

Accordingly, the first member 200 of the present disclosure may be vertically moved forward by the applicator.

Additionally, the microneedle 500 may be vertically separated from the first member 200 by the motion of the first member 200 moving vertically forward. The microneedle 500 may be vertically separated from the first member 200 and attached in a direction perpendicular to the front of the applicator.

For example, the case fastening part 220 may have a shape that extends rearward by a predetermined length. At this time, a guide groove 120 is formed on the inner side of the case 100 into which the case fastening part 220 may be guided and inserted so that the case fastening part 220 may be inserted into the inner side of the case 100 while maintaining a consistent direction.

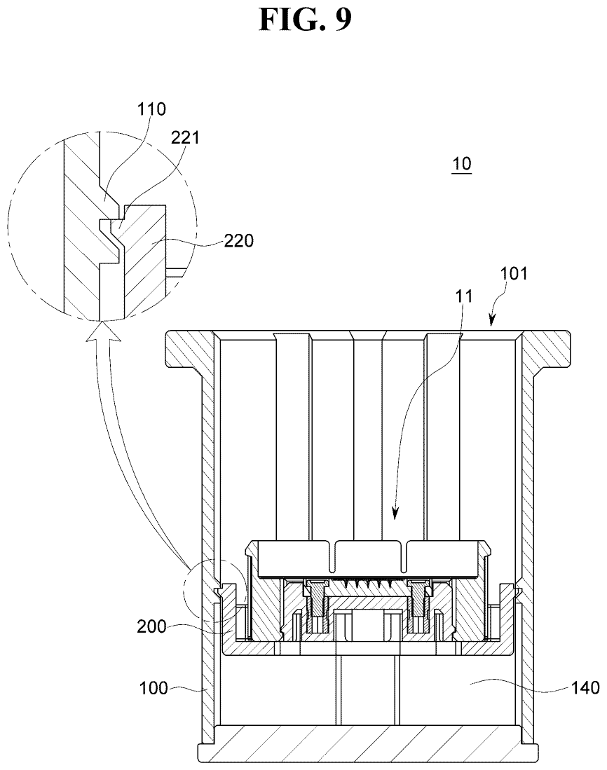

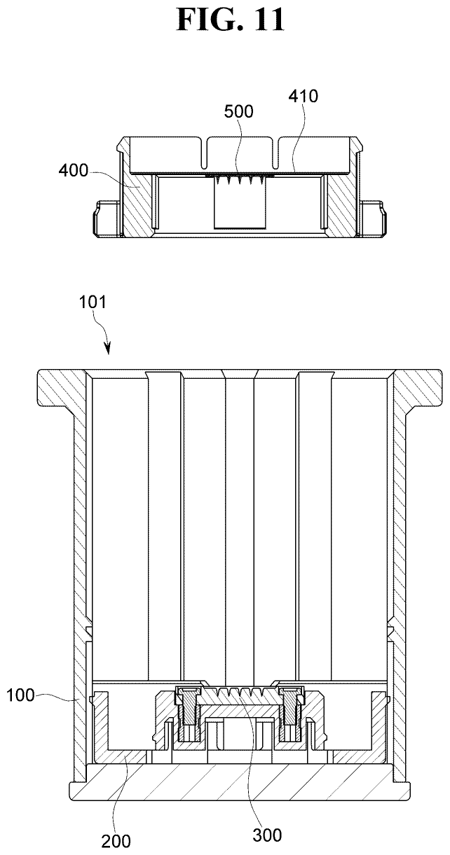

For example, the case fastening part 220 is provided with an outwardly protruding protrusion 221, and a catching projection (110 in FIG. 9) into which the protrusion 221 may be inserted is provided on the inner side of the case 100, so that the case fastening part 220 may be fixed at a predetermined position within the case 100 in a state in which rotation and longitudinal directional movement are limited. This will be described in detail with reference to FIGS. 9 and 11 described later.

The mold 300 is provided with a microneedle forming part 310 at the center of the mold 300 for forming the microneedle 500.

The microneedle forming part 310 is provided with a plurality of grooves having a predetermined shape so that the microneedle 500 may be formed or mounted.

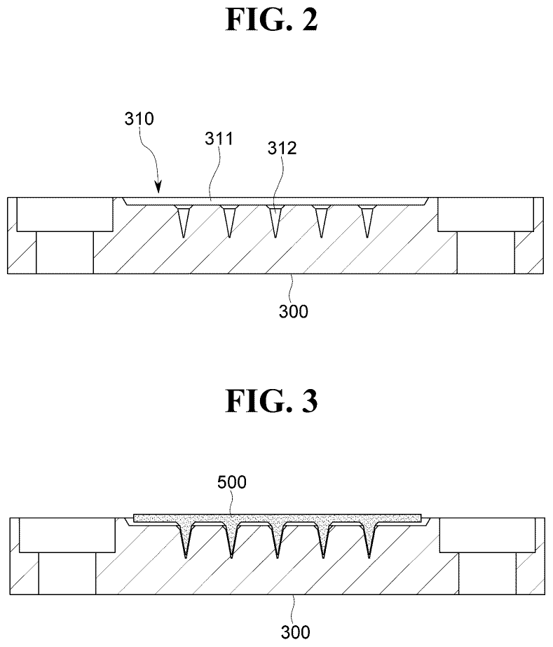

For example, a chemical may be filled into the microneedle forming part 310 and then cured to form a microneedle 500. Accordingly, the microneedle 500 may be stably formed within the mold, the microneedle 500 may be prevented from being exposed to the outside, and the microneedle 500 may be safely protected from impact. This will be described in detail with reference to FIGS. 2, 3, and 4 described later.

For example, the mold 300 may be manufactured from a metal material or a polymer material, and for example, a plastic material may be applied. Accordingly, the mold 300 may be manufactured at an inexpensive unit price, sterilization treatment may be facilitated, and the microneedle 500 formed in the microneedle forming part 310 may be easily separated from the microneedle forming part 310.

The second member 400 is for guiding the forward insertion of the applicator, and is disposed on the rear surface of the first member 200.

The second member 400 may be provided with an adhesive means 410 and a guide member 420 which is formed to surround the adhesive means 410 and extends rearward by a predetermined length.

The second member 400 may be coupled to and separated from the first member 200, and may be coupled to the applicator to be separated from the first member 200 together with the microneedle 500. This will be described in detail with reference to FIGS. 7 and 8 described later.

The second member 400 is provided in a state coupled to the first member 200, but when applied to the applicator, the microneedle 500 may be separated from the first member 200 in a state in which they are attached to the second member 400, thereby preventing contamination and damage to the microneedle 500 before use and improving convenience of use.

For example, when the second member 400 and the first member 200 are fastened, the front surface of the adhesive means 410 located at the central portion of the second member 400 and the rear surface of the microneedle 500 formed in the mold 300 mounted on the mold mounting part 210 come into contact with each other. Accordingly, the microneedle 500 formed in the mold 300 in the microneedle package 10 may be isolated from the external environment by the mold 300, the first member 200, and the adhesive means 410, thereby preventing contamination and damage. This will be described in detail with reference to FIGS. 7 and 8 described later.

In the microneedle package 10 of the present disclosure, the mold mounting part 210, the mold 300, the microneedle 500, and the adhesive means 410 are each disposed longitudinally in a continuous manner in a state in which they each have a front surface and a rear surface having a predetermined area in the thickness direction, and the applicator may be longitudinally inserted into the case 100, thereby having a structure in which the rear surface of the microneedle 500 is firmly attached to the front surface of the adhesive means 410, and the rear surface of the adhesive means 410 is firmly attached to the front surface of the applicator pressing part.

In this way, the present disclosure provides the second member 400 in a state of being coupled to the first member 200, but when applied to the applicator, the microneedle 500 may be separated from the first member 200 in a state in which the microneedle 500 is adhered to the adhesive means 410, thereby preventing contamination and damage to the microneedle 500 before use and improving convenience of use.

In addition, the first member 200 and the second member 400 of the present disclosure are installed in a case 100 that extends in the longitudinal direction to have a predetermined length, and the first member 200 is fixed in the case 100, but a structure in which the second member 400 is not fixed in the case 100 may be applied so that the second member 400 is easily separated from the case 100 by the applicator.

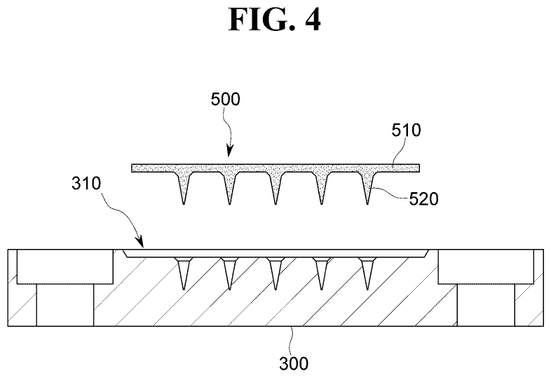

FIGS. 2, 3, and 4 illustrate embodiments in which a microneedle is formed and separated in a mold according to an embodiment of the present disclosure, FIG. 2 illustrates a state before the microneedle 500 is formed in a microneedle forming part 310, FIG. 3 illustrates a state in which the microneedle 500 is formed in a microneedle forming part 310, and FIG. 4 illustrates an embodiment in which the microneedle 500 is separated from a microneedle forming part 310.

Referring to FIG. 2, a microneedle forming part 310 may be provided in the mold 300.

For example, the microneedle forming part 310 may include a base forming part 311 formed by recessing a predetermined depth in the central portion of the mold 300 and a plurality of needle forming parts 312 extending forward from the base forming part 311 by a predetermined depth.

Referring to FIGS. 3 and 4, the microneedle 500 may be formed in the microneedle forming part 310.

For example, when applying a chemical for manufacturing a soluble microneedle to the microneedle forming part 310, the chemical may be filled into the microneedle forming part 310, thereby forming a base 510 in the base forming part 311 and forming a needle 520 in the needle forming part 312.

Referring to FIG. 4, since the entire surface of the microneedle 500 is simultaneously separated longitudinally from the mold 300 along with an adhesive means (not shown), the needles 520 may be formed vertically from the base 510 without being bent.

In this way, until the microneedle 500 is separated from the mold 300 using the applicator, the microneedle 500 of the present disclosure may be prevented from exposure to the external environment and safely protected from impact, and at the same time, the needles 520 may be formed vertically from the base 510.

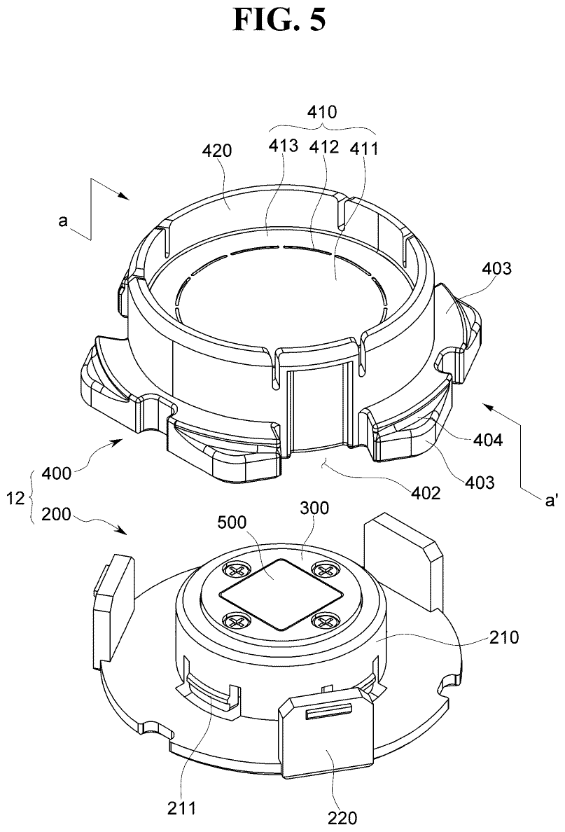

FIGS. 5 and 6 illustrate a first member and a second member according to an embodiment of the present disclosure, FIG. 6 illustrates a cut cross-section taken along the aa′ direction in FIG. 5, and FIGS. 7 and 8 illustrate an embodiment of a microneedle cartridge and a second member separated therefrom according to an embodiment of the present disclosure, wherein FIG. 7 illustrates one embodiment of a microneedle cartridge, and FIG. 8 illustrates that the second member and the microneedle are separated in FIG. 7.

Referring to FIGS. 5, 6, 7, and 8, the second member 400 is provided in a state of being seated on the first member 200, and the second member 400 may be separated from the first member 200 by the insertion motion of the applicator.

The second member 400 may be provided with a guide member 420 that defines a space for guiding the approach of the applicator. For example, the guide member 420 may be formed by extending a predetermined length rearward from the second member 400. For example, the distal end of the applicator may be guided to surround the guide member 420. For example, the guide member 420 may be composed of a plurality of segments separated by predetermined length units. Accordingly, the applicator is guided by the guide member 420 to vertically contact the rear surface of the microneedle 500 disposed in the mold 300 and the rear surface of the adhesive means 410, and when the distal end of the applicator is inserted into the second member 400, a predetermined elastic force by the guide member 420 is applied so that the applicator is advanced in the forward direction until the moment when the applicator and the adhesive means 410 come into contact, and the applicator may be fastened to the distal end of the applicator with the predetermined elastic force.

The second member 400 may be provided with a plurality of tongue parts 403 protruding outward in the thickness direction from the guide member 420.

A case fastening part guide part 402 into which the case fastening part 220 may be inserted may be provided between the plurality of tongue parts 403. Accordingly, the second member 400 may be vertically inserted and withdrawn from the rear surface of the first member 200 so that the needles 520 may be formed vertically from the base 510. In addition, the case fastening part 220 is inserted into the case fastening part guide part 402 so that the rotation of the second member 400 may be restricted.

The front surfaces of the tongue parts 403 are the surfaces that come into contact with the skin when the microneedle 500 is fired onto the skin, and are formed to have a predetermined area so that the position and direction of the applicator on the skin may be stably set.

A tongue groove 404 may be provided on the tongue parts 403. The distal end of the applicator may be fitting-coupled to the tongue groove 404, and as the applicator is withdrawn, the second member 400 may be separated from the first member 200.

The second member 400 may be provided with a step 430 formed by extending a predetermined length inward from the guide member 420.

The step 430 may protrude to the inner side of the guide member 420 at a predetermined height of the second member 400 and extend forward of the second member 400 to define a region into which the first member 200 is inserted.

An adhesive means 410 may be seated on the step 430, and in one embodiment, a thickness directional distal end of the adhesive means 410 may be adhered to the step surface of the step 430.

The adhesive means 410 may include a needle attachment part 411, a second member attachment part 413, and an adhesive means cutting line 412.

The needle attachment part 411 is a portion that contacts the microneedle 500 and may have adhesive force on the front surface. Accordingly, the rear surface of the microneedle 500 may be adhered to the front surface of the needle attachment part 411.

When the microneedle 500 is launched into a target area, for example, the skin, the needle attachment part 411 may be separated from the second member attachment part 413.

Furthermore, when the microneedle 500 attached to the adhesive means 410 is brought into contact with the skin using an applicator, the adhesive means 410 may be adhered to the skin by the adhesive force of the region of the needle attachment part 411 where the microneedle 500 is not attached.

The second member attachment part 413 may include a region attached to the step 430 of the adhesive means 410. The adhesive means 410 may be secured to the second member 400 by the second member attachment part 413.

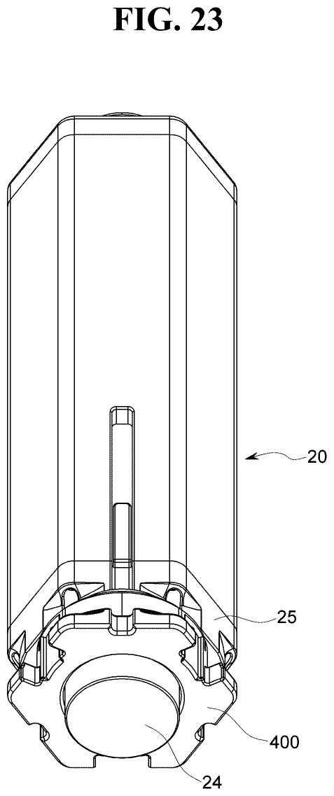

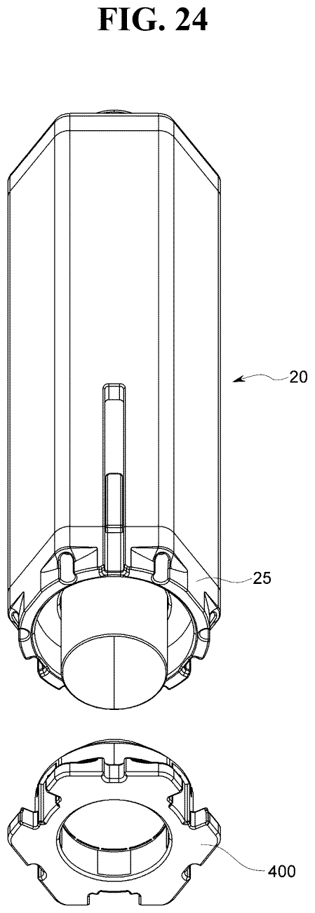

The adhesive means cutting line 412 may be defined as a void region formed between the needle attachment part 411 and the second member attachment part 413. Accordingly, when the applicator presses the needle attachment part 411, the needle attachment part 411 may be easily separated from the second member attachment part 413 along the adhesive means cutting line 412. Details will be described with reference to FIGS. 23 and 24 described later.

The second member 400 may have second member penetration parts 401a and 401b formed by passing through the lower and upper portions, and the second member penetration parts 401a and 401b may have different vertical directional cross-sectional areas based on the adhesive means 410. For example, the second member penetration part 401b located at the rear of the second member 400 has a vertical directional cross-sectional area defined by the aforementioned guide member 420, and the second member penetration part 401a located at the lower portion of the second member 400 is defined as an area narrower than the second member penetration part 401b as much as the step 430.

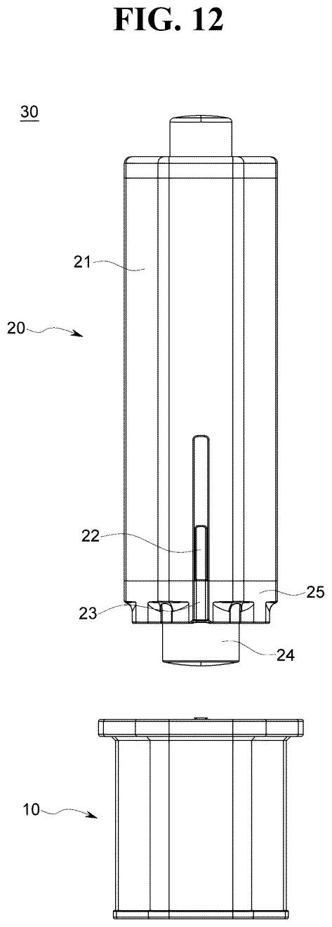

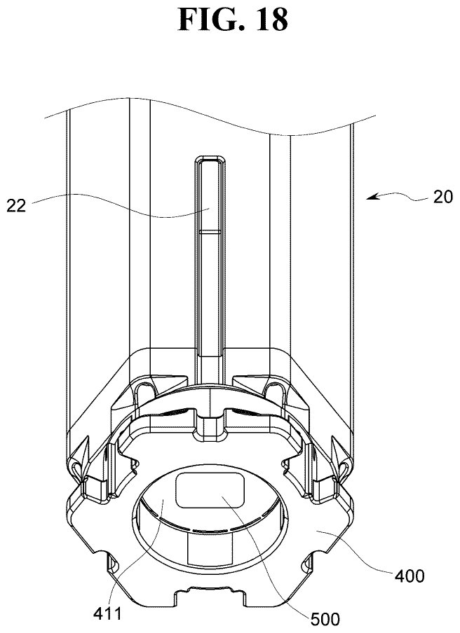

At this time, the pressing part (FIGS. 12 and 24) of the applicator described later inserts along the adhesive means housing penetration part 401b and presses the needle attachment part 411 to attach the rear surface of the microneedle 500 to the front surface of the needle attachment part 411, and when the pressing part returns to the rear in a state in which the rear surface of the needle attachment part 411 is attached to the pressing part, the needle attachment part 411 is separated from the second member attachment part 413 while the cutting line 412 is being cut. This will be described in detail with reference to FIGS. 18, 19, and 20 described later.

Referring to FIGS. 7 and 8, a structure in which the first member 200 and the second member 400 are coupled and separated from each other in the microneedle cartridge 11 may be applied. That is, the microneedle cartridge 11 may be provided in a state in which the first member 200 and the second member 400 are coupled, and a structure may be applied in which the second member 400 and the microneedle 500 are separated from the microneedle cartridge 11 as the second member 400 is separated from the first member 200 by the applicator.

The microneedle cartridge 11 may be provided in a state in which the first member 200 and the second member 400 are coupled to each other. Accordingly, the rear of the microneedle 500 formed in the mold 300 fixed to the mold mounting part 210 is covered by the adhesive means 410, thereby blocking the microneedle 500 from being exposed to the outside. Accordingly, contamination and damage to the microneedle 500 are prevented.

Referring to FIG. 7, the microneedle cartridge 11 may include a state in which the first member 200 and the second member 400 are coupled. Referring to FIG. 8, the first member 200 and the second member 400 may be separated from each other by the withdrawal action of the applicator (not shown). At this time, the mold 300 fixed to the mold mounting part 210 is remained in a state in which it is fixed to the mold mounting part 210, and the microneedle 500 formed in the mold 300 may be separated from the mold 300 in a state in which the microneedle 500 is attached to the adhesive means 410 of the second member 400.

For example, when the applicator is fastened to the microneedle cartridge 11, the second member 400 is coupled to the applicator, and when the applicator is separated from the microneedle cartridge 11, the second member 400 may be separated from the first member 200. Accordingly, the microneedle 500 attached to the adhesive means 410 may also be vertically extracted from the mold 300 and safely separated without damaging the microstructure of the microneedle 500. In addition, since there is no need for the operator to contact the region where the microneedle 500 is mounted, damage and contamination of the microneedle 500 are prevented.

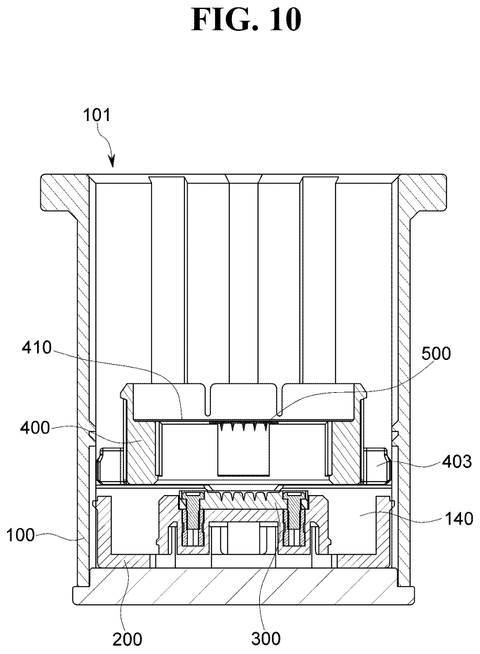

FIGS. 9, 10, and 11 show embodiments in which a microneedle cartridge is mounted and separated from a case in a microneedle package according to an embodiment of the present disclosure. FIG. 9 shows a state in which a microneedle cartridge is mounted in a case, FIG. 10 shows a state in which a first member moves forward from a second member in a case due to the insertion of an applicator, and FIG. 11 shows a state in which the second member is withdrawn from the case along with the withdrawal of the applicator after the operation of FIG. 10. Meanwhile, the illustration of the applicator is omitted in FIGS. 9, 10, and 11.

Referring to FIG. 9, a microneedle cartridge 11 may be fastened to a case 100.

The microneedle cartridge 11 may be inserted into the case opening 101 of the case 100 and secured at a predetermined location within the case 100.

The case fastening part 220 of the first member 200 may be provided with an outwardly protruding protrusion 221. Correspondingly, a catching projection 110 may be provided on the inner side of the case 100, the protrusion 221 may engage with the catching projection 110, thereby securing and coupling the microneedle cartridge 11 at a predetermined location to restrict rotation and longitudinal movement. At this time, the first member 200 may be detachably secured to the catching projection 110. Accordingly, the microneedle package 10 may be fixed in position so that the distal end of the applicator approaches in a direction perpendicular to the adhesive means and the base of the microneedle, thereby allowing the needles in the microneedle to be formed in a direction perpendicular to the base.

For example, the second member 400 may be fixed in position by being seated on a stepped projection 140 provided within the case 100.

At this time, the second member 400 and the first member 200 may be installed so as to overlap within the case 100.

In addition, the microneedle package 10 of the present disclosure may be stored in a state in which the case opening 101 is closed by a sealing member (not shown) or the like in the state of FIG. 9. Accordingly, contamination of the microneedle cartridge 11 during its storage period can be prevented, and the convenience of storage can be improved.

Referring to FIG. 10, the first member 200 may move forward from the second member 400 in the case 100 due to the insertion of the applicator, thereby separating the second member 400 and the microneedle 500 from the first member 200.

For example, when the applicator is inserted forward into the case opening 101 along the longitudinal direction of the case 100, the first member 200 is moved forward by the applicator and is seated on the bottom surface of the case 100, and the second member 400 is prevented from being pushed since the tongue parts 403 are caught on the stepped projection 140. Accordingly, the microneedle may be stably attached to the front of the applicator.

Referring to FIG. 11, when the applicator is withdrawn in a vertical direction from the case 100, the first member 200 may maintain a state in which it is seated on the bottom surface of the case 100 as it is, and the second member 400 may be extracted to the outside of the case 100 in a state in which it is mounted to the front of the applicator.

FIGS. 12, 13, and 14 show an applicator package including an applicator and a microneedle package according to an embodiment of the present disclosure, and FIGS. 15, 16, 17, 18, 19, and 20 show embodiments of the applicator package according to an embodiment of the present disclosure according to the order of contact with the skin, microneedle firing, and detachment, wherein FIGS. 15, 16, and 17 show shapes of the applicator package, and FIGS. 18, 19, and 20 show lower portions of the applicator package, respectively.

Hereinafter, FIGS. 12, 13, 14, 15, 16, 17, 18, 19, and 20 will be described.

The applicator package 30 may include six operating states.

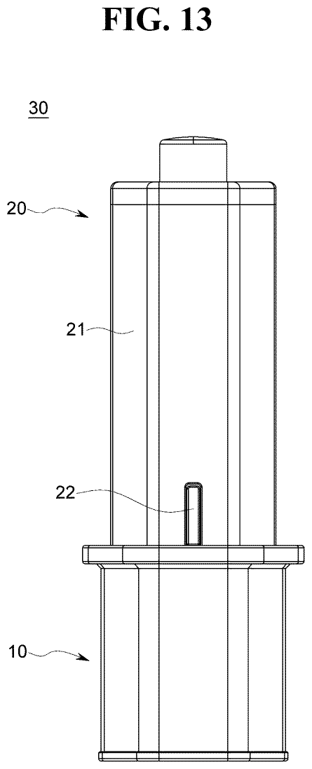

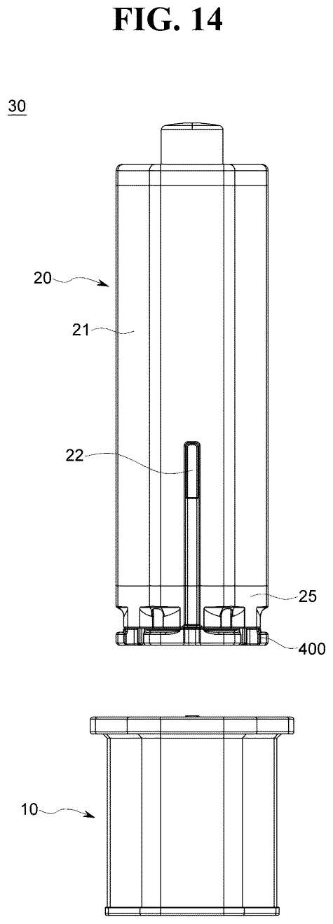

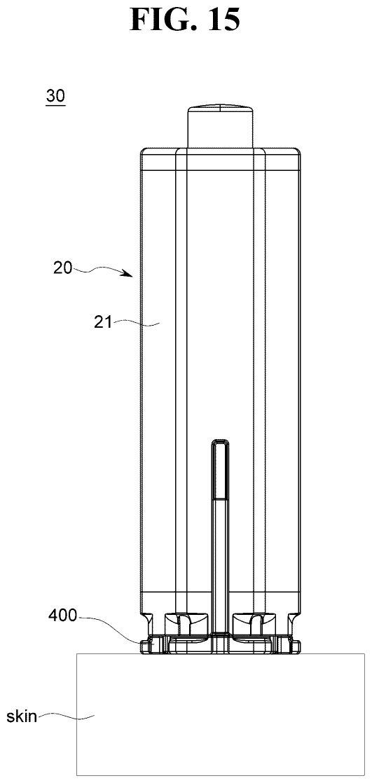

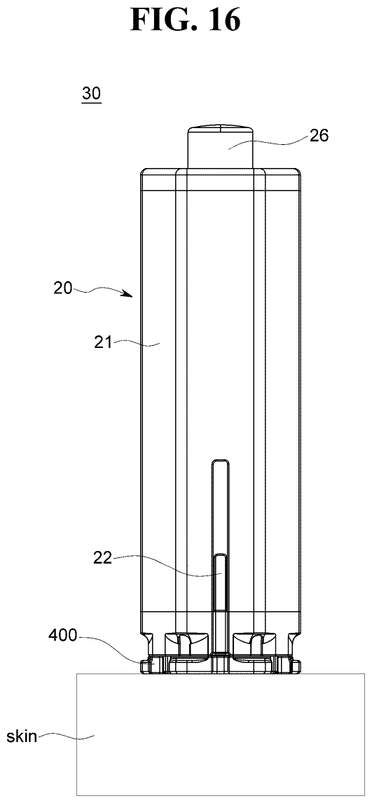

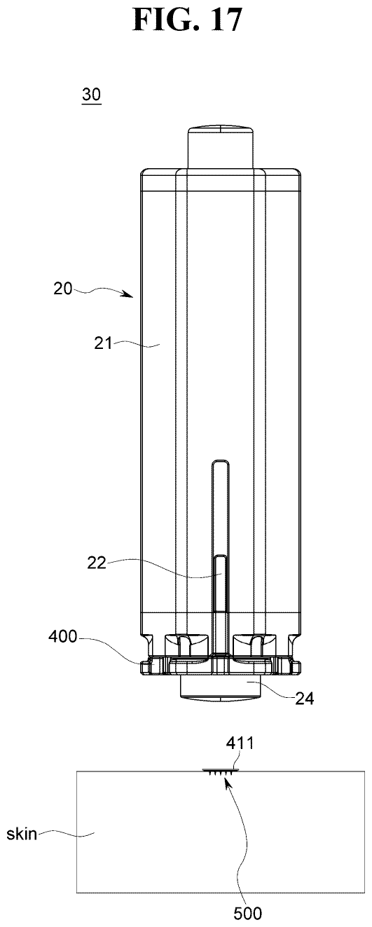

FIG. 12 shows a first operating state in which an applicator 20 is inserted into a microneedle package 10, FIG. 13 shows a second operating state in which a microneedle package 10 is fastened to an applicator 20, FIG. 14 shows a third operating state in which an applicator 20 is separated from a microneedle package 10, FIG. 15 shows a fourth operating state in which an applicator package 30 according to the third operating state is brought into contact with the skin, FIG. 16 shows a fifth operating state in which a microneedle is fired from an applicator package 30 brought into contact with the skin, and FIG. 17 shows a sixth operating state in which the applicator package 30 is detached from the skin. Meanwhile, FIG. 9 described above shows the state of FIG. 12, FIG. 10 shows the state of FIG. 13, and FIG. 11 shows the state of FIG. 14, respectively.

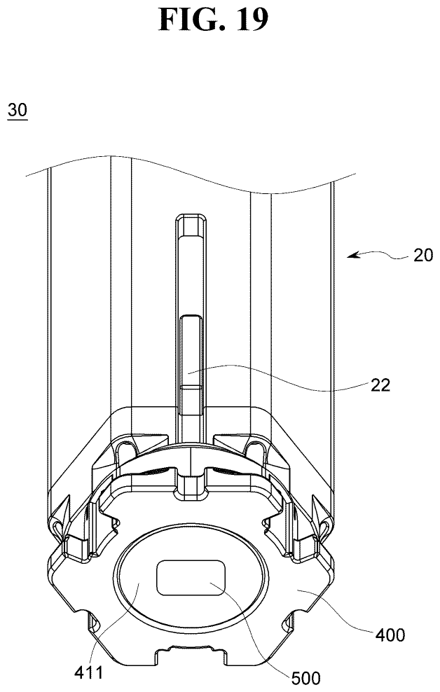

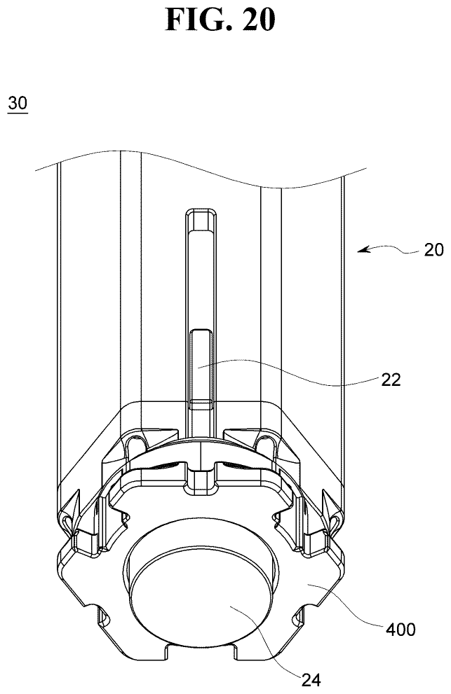

FIG. 18 shows the front of the applicator package in the fourth operating state, FIG. 19 shows the front of the applicator package in the fifth operating state, and FIG. 20 shows the front of the applicator package in the sixth operating state, respectively.

The applicator 20 may include an applicator body 21, a slide 22, a pressing part 24, a second member fastening part 25, and a shooting button 26.

The applicator body 21 may have be held by the user to insert its distal end into the microneedle package 10, thereby having a form in which the applicator body 21 is extended by a predetermined length in the longitudinal direction.

The slide 22 may be provided at the distal end of the applicator body 21. For example, the slide 22 may move a predetermined distance from the distal end of the body 21.

For example, the slide 22 may move up and down along a linear guide 23 formed longitudinally from the side surface of the applicator body 21 and may be fixed at a predetermined position.

For example, the linear guide 23 may be a groove form provided longitudinally on the side surface of the applicator body 21. For example, when the applicator 20 performs an operation of inserting into the case 100, a plurality of protrusion pieces 130 provided on the case 100 may be inserted into the linear guides (23) to move longitudinally while restricting the rotation of the applicator 20, thereby allowing the applicator 20 to be inserted vertically into the microneedle 500.

For example, at least one linear guide 23 may be a groove form provided longitudinally on the inner side of the applicator body 21. For example, at least one protrusion piece 130 may include a form that protrudes rearwardly along the longitudinal direction from the bottom surface of the case 100.

For example, an operation of being pushed rearward by the protrusion pieces 130 provided on the inner side of the case 100 may be performed on the slide 22.

For example, as the applicator 20 is inserted into the microneedle package 10, the slide 22 may be pushed rearward, and the slide 22 may be fixed in a rearwardly pulled state at a depth where the second member 400 and the microneedle 500 are mounted at the end of the applicator body 21 within the case 100.

For example, when the applicator 20 is inserted into the microneedle package 10, the protrusion pieces 130 provided in the inner side of the case 100 may be inserted into the linear guide 23, so that the applicator 20 is inserted into the case 100 in a vertical direction with respect to the microneedle 500, thereby forming the microneedle in a vertical direction on the front surface of the applicator without being bent.

For example, one or more slides 22 may be provided, and for example, three are provided in different regions on the side surface of the applicator 20, but the protrusion pieces 130 are provided at positions corresponding to the slides 22, so that the applicator 20 may be stably inserted into the case 100 along the vertical direction with respect to the microneedle 500.

At least a portion of the pressing part 24 may be provided within the applicator body 21, thereby moving longitudinally along the space formed within the applicator body 21. For example, the pressing part 24 may be mechanically or electrically driven using a spring, hydraulic pressure, a driving motor, a gear motion, or the like within the applicator body 21, but the present disclosure is not limited thereto.

At this time, the slide 22 and the pressing part 24 can move together in the same direction along the longitudinal direction through physical or electrical linkage, and can move in the direction in which the microneedle 500 is disposed at a preset constant speed and pressure.

The second member fastening part 25 is formed at the distal end of the applicator body 21 and may be fastened to the second member 400.

The shooting button 26 may be provided on the outer side of the applicator body 21 and may be a start button that causes the pressing part 24 to advance forward. The shooting button 26 and the pressing part 24 may have their operations mechanically or electrically linked by a spring, hydraulic pressure, a driving motor, a gear motion, or the like. For example, when a user touches or applies pressure to the shooting button 26, the pressing part 24 may move forward at a preset pressure. Meanwhile, the slide 22 may move forward together with the pressing part 24, and at this time, the movement length of the slide 22 may be controlled to prevent the end of the slide 22 from impacting the skin, or a structure in which the length of the slide 22 is set may be applied. For example, the shooting button 26 may be formed to protrude from the rear or the side portion of the applicator body 21.

The first operating state illustrated in FIG. 12 may include the applicator 20 approaching the microneedle package 10 in a state in which the slide 22 is positioned a predetermined distance spaced from the end of the applicator 21 and the pressing part 24 protrudes from the end of the applicator body 21. At this time, as the applicator 20 is inserted into the case 100 of the microneedle package 10, the slide 22 is caught on the edge of the opening of the case 100, but a structure in which the second member fastening part 25 and the end of the applicator body 21 are inserted into the case 100 is applied, so that the pressing part 24 is pulled rearward together with the slide 22.

The second operating state illustrated in FIG. 13 may include that the applicator 20 is inserted into the case 100 of the microneedle package 10 to a preset depth, and the second member 400 is fastened to the second member fastening part 25 disposed at the distal end of the applicator 20. At this time, they may be fixed in position by a spring, hydraulic pressure, a driving motor, a gear movement, or the like in a state in which the slide 22 and the pressing part 24 may be pulled to the rearmost position.

At this time, the pressing part 24 may move rearward together with the slide 22 so as not to touch the adhesive means (not shown) disposed on the second member 400. Accordingly, a structure may be applied in which the needle attachment part (not shown) is not pre-separated from the adhesive means (not shown) by the pressing part 24.

In another embodiment, a structure may be applied in which the pressing part 24 applies a predetermined pressure to the needle attachment part (not shown) from the adhesive means (not shown) disposed on the second member 400 so that the microneedle (not shown) is sufficiently adhered to the front surface of the needle attachment part (not shown).

The third operating state illustrated in FIG. 14 may include that the applicator 20 is withdrawn from the microneedle package 10 to separate the applicator 20 from the microneedle package 10 in a state in which the second member 400 is fastened to the second member fastening part 25. At this time, a state in which the microneedle (not shown) is attached to the front surface of the needle attachment part (not shown) disposed on the second member 400 may be included.

The fourth operating state illustrated in FIG. 15 may include that the applicator package 30 according to the third operating state is contacted with the skin. At this time, the front surface of the tongue part (not shown) of the second member 400 may contact the skin.

The fifth operating state illustrated in FIG. 16 may include that the microneedle 500 from the applicator package 30 in contact with the skin is fired to the skin, and the pressing part 24 and the slide 22 move forward by the operation of the shooting button 26.

At this time, as the pressing part 24 moves forward along the longitudinal direction from the applicator 20, the needle attachment part (not shown) may be pressed forward, thereby separating the needle attachment part (not shown) from the second member attachment part (not shown) while cutting out the adhesive means cutting line (not shown). With this operation, the microneedle 500 attached to the front surface of the needle attachment part (not shown) may be attached to the skin.

Referring to FIG. 18, which shows the front of the applicator package 30 in the fourth operating state, and FIG. 19, which shows the front of the applicator package 30 in the fifth operating state, the position of the needle attachment part 411 may be located in rear compared to the front surface of the second member 400, thereby having a structure in which the microneedle 500 does not contact the skin before the shooting button (not shown) is pressed after the applicator package 30 comes into contact with the skin. Accordingly, the applicator package 30 of the present disclosure may attach the microneedle 500 to the skin at the regular position and attach it to the skin in a direction perpendicular to the skin, thereby attaching the microneedle 500 formed in the mold (not shown) in a form as it is to the skin.

The sixth operating state illustrated in FIG. 17 may include that the applicator package 30 is detached from the skin after firing of the microneedle 500 has been completed. At this time, the slide 22 and the pressing part 24 may return to the first operating state.

Referring to FIG. 20, which illustrates the front of the applicator package 30 in the sixth operating state, a structure may be applied in which the needle attachment part 411 and the microneedle 500 are separated from the front of the pressing part 24, and the slide 22 and the pressing part 24 return to the position of the first operating state.

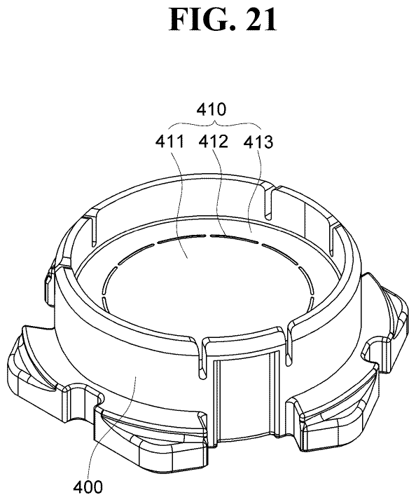

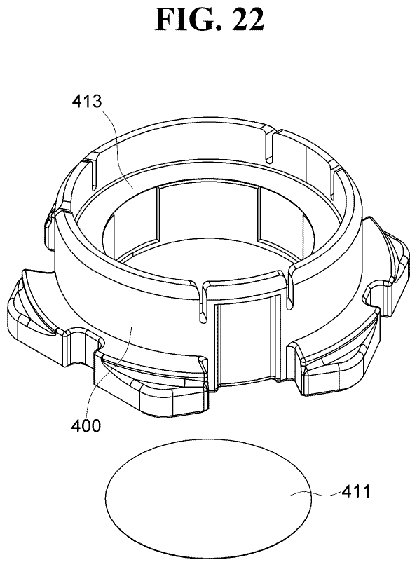

FIGS. 21 and 22 show embodiments in which the needle attachment part is detached from the second member according to an embodiment of the present disclosure.

Referring to FIG. 21, an adhesive means 410 may be disposed on the second member 400.

The adhesive means 410 may include a needle attachment part 411, a second member attachment part 413, and an adhesive means cutting line 412, and the structure and effects thereof may be applied as described above.

Referring to FIG. 22, the needle attachment part 411 to the front surface of which the microneedle 500 is attached may be separated from the second member attachment part 413 and attached to the skin by the pressing action of the pressing part (not shown).

FIGS. 23 and 24 show embodiments in which the second member is separated from an applicator according to an embodiment of the present disclosure.

Referring to FIG. 23, the second member 400 may be in a state in which the second member 400 is fastened to the second member fastening part 25 provided in front of the applicator 20 after the sixth operating state.

Referring to FIG. 24, the second member 400 may be separated from the second member fastening part 25 of the applicator 20. Accordingly, a new microneedle package may be coupled to the applicator 20 to fasten another second member 400, thereby reusing the applicator 20.

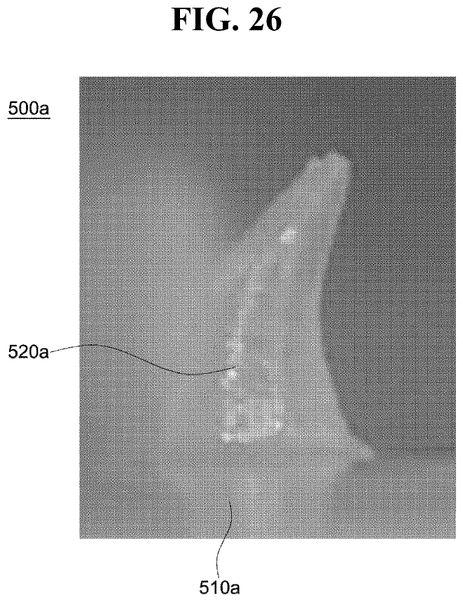

FIGS. 25 and 26 show forms of a microneedle, wherein FIG. 25 shows a microneedle manufactured according to one embodiment of the present disclosure, and FIG. 26 shows a conventionally manufactured microneedle.

Referring to FIGS. 25 and 26, the conventionally manufactured microneedle 500a is formed in a form in which the needle 520a is bent from the base 510a, but it can be confirmed that the microneedle 500 manufactured according to one embodiment of the present disclosure is formed in a form in which the center line of the needles 520 is vertically extended from the base 510, and the area of the cross section gradually narrows at a predetermined ratio as it extends from the base 510.

As described above, Specific description for the present disclosure has been made by embodiments with reference to the attached drawings, but since the above-described embodiments merely explain preferred examples of the present disclosure, the present disclosure should not be construed as being limited to the above-described embodiments, and the scope of rights of the present disclosure should be understood as the claims described later and concepts equivalent thereto.

For example, the drawings schematically show each component as the main body to aid understanding, and the thickness, length, number, etc. of each component illustrated may differ from the actual configuration due to the progress of drawing up of the drawing. Furthermore, the material, shape, dimensions, etc. of each component shown in the above-described embodiments are merely examples and are not particularly limited, and various modifications are possible without substantially departing from the effects of the present disclosure.

### DESCRIPTION OF REFERENCE NUMERALS

10: Microneedle package 11: Microneedle cartridge 12: Needle protection part
20: Applicator 21: Applicator body 22: Slide 23: Linear guide
24: Pressing part 25: Second member fastening part 26: Shooting button 30: Applicator package
100: Case 101: Case opening 110: Catching projection 120: Guide groove 130: Protrusion piece
140: Stepped projection 200: First member 210: Mold mounting part 211: Elastic piece 220: Case fastening part
221: Protrusion 300: Mold 310: Microneedle forming part 311: Base forming part
312: Needle forming part 400: Second member 401a, 401b: Second member penetration part
402: Case fastening part guide part 403: Tongue part 404: Tongue groove 410: Adhesive means
411: Needle attachment part 412: Adhesive means cutting line 413: Second member attachment part 420: Guide member
430: Step 500, 500a: Microneedle 510, 510a: Base 520, 520a: Needle
521: Needle centerline

1. A microneedle package comprising:
a needle protection part in which a microneedle is disposed; and
a case in which the needle protection part is disposed and which guides an applicator to be seated on the microneedle.

2. The microneedle package of claim 1, wherein the needle protection part comprises a case fastening part in the form of extending rearward.

3. The microneedle package of claim 2, wherein the needle protection part further comprises a first member provided with the case fastening part and a second member for being coupled to the rear of the first member, and the case fastening part allows the position of the first member to be fixed at a predetermined position within the case, and the first member moves forward by an operation in which the case fastening part is pressed by the applicator.

4. The microneedle package of claim 3, wherein the case fastening part is provided with three pressed regions that are pressed by the applicator, and the first member is pressed by the applicator so that the first member has a surface perpendicular to the applicator, thereby forming the microneedle vertically.

5. The microneedle package of claim 4, wherein the microneedle is vertically separated from the first member by an operation of the first member moving vertically forward.

6. The microneedle package of claim 1, wherein the needle protection part further comprises a first member and a second member for being coupled to the rear of the first member, and the second member is provided in a state coupled to the first member, but when applied to the applicator, the microneedle is separated from the first member in a state in which the microneedle is attached to the second member, thereby preventing contamination and damage to the microneedle before use and improving convenience of use.

7. The microneedle package of claim 6, wherein the first member and the second member are installed within a case extending in the longitudinal direction to have a predetermined length, but the second member is seated on a stepped projection provided at the bottom of the case, and the first member is detachably fitting-coupled to be fixed inside the case, thereby overlappingly installing the second member and the first member within the case.

8. The microneedle package of claim 7, wherein when the applicator is inserted into the case, the second member is fastened to the front portion of the applicator in a state in which the second member is seated on the stepped projection, and the first member moves to the bottom surface of the case by pressing of the applicator.

9. The microneedle package of claim 1, wherein the needle protection part is provided with a mold mounting part for seating the mold, and the mold is provided with a microneedle forming part recessed to a predetermined depth on the rear surface.

10. The microneedle package of claim 9, wherein the microneedle forming part comprises a base forming part and a plurality of needle forming parts extending forward from the base forming part.

11. The microneedle package of claim 6, wherein the adhesive means having adhesive force is disposed forwardly on the second member, the adhesive means comprises: a needle attachment part which is formed in the center and is for adhering the microneedle; a second member attachment part which is defined as a region where the microneedle is seated on the second member; and an adhesive means cutting line which is defined as an empty region formed between the needle attachment part and the second member attachment part, and wherein the needle attachment part is separated from the second member attachment part when the microneedle is fired toward the target region.

12. The microneedle package of claim 11, wherein the second member comprises: a guide member extending to the rear of the second member to define a space that guides the approach of the applicator; and a step protruding to the inside of the guide member at a predetermined height and extending to the front of the second member to define a region into which the first member is inserted, and the second member attachment part is disposed on a step surface of the step.

13. The microneedle package of claim 12, wherein the guide member is divided into a plurality of pieces to impart elasticity when the applicator is inserted, thereby imparting a fixing force to the applicator.

14. The microneedle package of claim 6, wherein the first member is provided with a plurality of case fastening parts that are in the form of extending rearward, and guide grooves for inserting the case fastening parts into the case while the case fastening parts are being guided through the guide grooves are provided on the inner side of the case, thereby easily installing the first member into the case, and restricting rotation of the first member after installation.

15. The microneedle package of claim 14, wherein the second member comprises a plurality of tongue parts which protrude outwardly so that the applicator is fastened to the tongue parts; and a case fastening part guide part defined as a space which is provided between the tongue parts and into which the case fastening parts are inserted, and wherein rotation of the second member from the first member is restricted by the case fastening part guide part.

16. The microneedle package of claim 1, wherein the needle protection part in which the microneedle is disposed is provided in a state in which the needle protection part is stored in a sealed space within the case, thereby preventing the inflow of foreign materials and preventing contamination of the microneedle prior to use.

17. An applicator package comprising:
the microneedle package of claim 3; and
an applicator coupled to the rear of the microneedle package.

18. The applicator package of claim 17, wherein during an operation of coupling the applicator to and then detaching it from the microneedle package, the second member is separated from the microneedle package and fastened to the applicator, but the microneedle is provided in front of the needle protection part.

19. The applicator package of claim 17, wherein the applicator comprises a pressing part that is at least partially inserted into the applicator and moves along the longitudinal direction of the applicator, and the rear surface of the microneedle is disposed in front of the pressing part.

20. The applicator package of claim 19, wherein the applicator further comprises a slide that is disposed at the front portion of the applicator and moves in the longitudinal direction, and the slide is operated in conjunction with the pressing part.

21. The applicator package of claim 20, wherein the applicator further comprises a linear guide provided at the side portion to allow the slide to move in the longitudinal direction.

22. The applicator package of claim 21, wherein the case further comprises protrusion pieces in such a form that they protrude to the inner side of the case, but extend in the longitudinal direction.

23. The applicator package of claim 22, wherein when the applicator is inserted into the case, the slide moves rearward of the applicator due to pressure applied by the protrusion pieces; the pressing part moves rearward of the applicator in conjunction with the movement of the slide; the slide and the pressing part that move rearward are fixed at a predetermined position; and after the slide and the pressing part are fixed at a predetermined position, the slide and the pressing part move forward so that the microneedle is separated from the needle protection part.
